SEQUENCE LISTING (10 sequences)

SEQ ID NO: 1 - DNA, 24 bp, Artificial Sequence
  synthetic primer
gttatcccactcctcagatcaagt

SEQ ID NO: 2 - DNA, 24 bp, Artificial Sequence
  synthetic primer
tcacagctacagtcatcacagtgt

SEQ ID NO: 3 - DNA, 20 bp, Artificial Sequence
  synthetic primer
gggaggaatcaccatcagaa

SEQ ID NO: 4 - DNA, 20 bp, Artificial Sequence
  synthetic primer
ctcaggtggaaatcgtggtt

SEQ ID NO: 5 - DNA, 20 bp, Artificial Sequence
  synthetic primer
cacagttgctgcctattcca

SEQ ID NO: 6 - DNA, 20 bp, Artificial Sequence
  synthetic primer
ggtgaggagccacctgagat

SEQ ID NO: 7 - DNA, 22 bp, Artificial Sequence
  synthetic primer
ggctgcatttgaacctgtatcc

SEQ ID NO: 8 - DNA, 24 bp, Artificial Sequence
  synthetic primer
gtgtctgcacaccctaaagaatcc

SEQ ID NO: 9 - DNA, 36 bp, Artificial Sequence
  synthetic primer
gatacggatccagacatcagggtgtcatggttggta

SEQ ID NO: 10 - DNA, 33 bp, Artificial Sequence
  synthetic primer
gatacaagcttatagcagagcttctccttgatg

(19) United States
(12) Patent Application Publication
(10) Pub. No.: US 2007/0294778 A1
(43) Pub. Date: Dec. 20, 2007

(54) Zebrafish model for assessing gastrointestinal motility

(76) Inventor: Adam Rich, Brockport, NY (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/724,741

(22) Filed: Mar. 16, 2007

Related U.S. Application Data
(60) Provisional application No. 60/782,993, filed on Mar. 16, 2006.

(51) Int. Cl. A01K 67/027

(52) U.S. Cl. 800/3

(57) ABSTRACT

The present invention has demonstrated the presence of the zebrafish KIT protein resulting from the expression of the kit gene in the zebrafish GI tract. As expression of the KIT protein is correlated with ICC in other species, the expression of the KIT protein in zebrafish indicates the presence of ICC in the zebrafish GI tract. ICC are required for spontaneous, coordinated contractions of GI smooth muscle. The present invention provides a zebrafish-based model system useful for elucidating the cellular and molecular mechanisms of gastrointestinal (GI) function and for identifying molecular targets for treating GI motility disorders in human.

### CROSS-REFERENCE TO RELATED APPLICATION

This application claims priority from U.S. Provisional Application No. 60/782,993, filed on Mar. 16, 2006.

### GOVERNMENT INTEREST

This invention was made in the course of work under grants sponsored in part by the National Institute of Health. Thus, the U.S. government has certain interests in the present invention.

### FIELD OF THE INVENTION

This invention relates to the use of zebrafish as a model to investigate gastrointestinal functions and to evaluate the effects of candidate compounds designed for treating gastrointestinal disorders in humans.

### BACKGROUND OF THE INVENTION

A large percentage of the population suffers from gastrointestinal (GI) motility disorders. Approximately 10-20% of the population suffers from irritable bowel syndrome, and 50% of patients with diabetes suffer from gastroparesis. Currently there are few effective drugs available for treating motility disorders, which results from a limited understanding of GI physiology at the molecular level and consequently a lack of drug targets.

Gastrointestinal motility is the coordinated contraction of smooth muscles resulting in mixing and ordered propulsion of the luminal contents. The coordinated contractions are driven by the electrical slow wave. It is believed that an intact network of interstitial cells of Cajal (ICC) is required for the generation and correct organization of the slow wave. ICC specifically and independently function as pacemaker cells, displaying a rhythmic and spontaneous electrical depolarization in resting membrane potential, thereby determining the frequency and amplitude of the electrical slow wave. ICC are located in a functionally critical position—they collect input from enteric nervous system, relay and transmit a signal to smooth muscles resulting in an electrical slow wave of a specific frequency and magnitude. However, there has been no identification of molecular targets located on ICC or specifically impacting ICC function. Current model systems, such as the murine model, do not allow direct visualization of GI motility in an intact, physiological setting, nor do they allow a direct assessment of experimental manipulation on ICC function and motility. Drug targets that modulate ICC function would appear to be ideal for the treatment of GI motility disorders. Hence there is a need for new model systems that permit assessment of GI functions in an intact, physiological setting in order to identify targets and develop effective drugs for treating GI disorders.

### SUMMARY OF THE INVENTION

The present inventor has established that the zebrafish GI tract contains all of the anatomical features that are known to control GI smooth muscle contraction in humans. Accordingly, the present invention provides a new model system to investigate gastrointestinal functions of humans and to evaluate the effects of candidate compounds designed for treating gastrointestinal disorders in humans.

In one embodiment, the present invention provides a method for determining the effect of a candidate compound designed to treat a gastrointestinal disorder in humans by administering the compound to a zebrafish or zebrafish larvae and assessing the effect of the compound on motility of the GI tract of the zebrafish or larvae.

In another embodiment, the present invention provides a method for identifying a compound that either promotes or inhibits GI motility by administering the compound to a zebrafish or zebrafish larvae, and comparing the GI transit time of a material in the zebrafish or larvae with that of a control zebrafish or larvae.

In still another embodiment, the present invention provides a method for assessing the side effect of a compound on gastrointestinal functions, wherein the compound has been developed for treating a non-gastrointestinal disorder. The method is based on evaluation of the motility of the GI tract of a zebrafish or zebrafish larvae that has been administered with the compound.

In another embodiment, the present invention provides a method for determining the function of a specific molecule or cellular component in the GI tract by obtaining a mutant zebrafish that lacks the molecule or cellular component, and evaluating the GI tract of the mutant zebrafish thereby determining the function of the molecule or cellular component.

In a further embodiment, the present invention provides a method for identifying the molecular or cellular target of a compound by determining the effect of the compound on the GI motility of a wild type zebrafish, and the lack of the effect of the compound on the GI motility in a mutant zebrafish, thereby identifying the molecular or cellular component that is dysfunctional in the mutant zebrafish as the target of the compound.

### BRIEF DESCRIPTION OF THE DRAWINGS

The file of this patent contains at least one drawing executed in color as determined by the U.S. Patent and Trademark Office. Copies of this patent with color drawing(s) will be provided by the Patent and Trademark Office upon request and payment of the necessary fees.

FIG. 1. kita mRNA is detectable in zebrafish GI tract.

FIG. 2. KIT-like immunoreactivity in the adult zebrafish GI tract.

FIG. 3. KIT-like immunoreactivity in zebrafish larvae specifically identifies cells in the GI tract. The image is a lateral view, oral is oriented to the left and dorsal is up. Scale bars indicate 50 am.

FIG. 4. Fluorescent images of KIT-like immunoreactivity (a), mitotracker red (b), PGP 9.5 (c), and synaptophysin (d) ACK2 immunoreactivity identifies cells in wholemounts and sections of the adult zebrafish GI tract.

FIG. 5. Direct observation of spontaneous GI contractions in zebrafish larvae.

FIG. 6. Spontaneous contractions are reduced and disorganized in the null kita mutant.

FIG. 7. RT-PCR amplifies the zebrafish Kit receptor tyrosine kinase kita and kitb, and Steel factor, kitla and kitlb, from isolated adult GI tract. Amplification products are shown for primer sets designed for each gene (Table 1), and for β-actin. Products of correct size for each primer set were observed and confirmed by sequencing.

FIGS. 8A-8B. The pigment pattern mutation Sparse, a kita null mutant, has decreased spontaneous GI contractions and an increased size of the GI tract. Optically transparent zebrafish larvae (7 dpf) were videotaped and contraction frequency was 0.59+/−0.05 contractions/minute (mean +/−SE, n=20) in wild type, and 0.33+/−0.03 contractions/minute (P<0.05, n=23) in homozygous Sparse mutant larvae. Cross-sectional area of the GI tract was measured in single frames for each experiment. Cross-sectional area was less for wild-type, 12,331+/−467, compared to the Sparse mutant larvae, 14,985+/−483 (P<0.05).

FIGS. 9A-9B. Spatiotemporal map results. Spatio temporal maps from control (FIG. 9A) and Gleevec®-treated (FIG. 9B) 11 dpf larvae. Spontaneous GI motility occurs at regular intervals and propagates along the GI tract at 11 dpf when ICC are fully present in the GI tract. Gleevec®, a tyrosine kinase inhibitor, blocks Kit function. Spontaneous motility was absent in Larvae treated with 50 μM Gleevec®. These data show that inhibiting Kit results in the loss of spontaneous propagating contractions, and suggest that ICC are necessary for coordinated and propagating GI contractions in zebrafish larvae.

FIG. 10. Spatiotemporal map results. Screen-shot of spatio temporal mapping program output. The top left panel is the map, which shows the converted image data. The top right panel shows a zebrafish larvae with a region of interest outlined.

### DETAILED DESCRIPTION OF THE INVENTION

Interstitial cells of Cajal (“ICC” or pacemaker cells) in the gastrointestinal (GI) tract of a number of mammals are thought to regulate, coordinate, and modulate the electrical slow wave (a spontaneous oscillation of the resting membrane potential) observed in the GI tract. The electrical slow wave controls contraction of GI smooth muscle cells. In other words, changes in the electrical slow wave underlie changes in GI motility.

The present inventor has identified for the first time a cell population within the zebrafish GI tract that expresses the zebrafish KIT protein, a marker for pacemaker cells or ICC, and thus has established that the zebrafish GI tract contains all of the anatomical features that are known to control GI smooth muscle contraction in humans. That is, like the human GI tract, the zebrafish GI tract includes KIT-positive ICC, enteric neurons, smooth muscle cells, epithelial cells and entero-endocrine cells. Accordingly, zebrafish can be used as a model system to investigate gastrointestinal functions of humans and to evaluate the effects of candidate compounds designed to treat gastrointestinal disorders in human.

As used herein, the term “kit” (in lower case) refers to the gene, while the term “KIT” refers to the protein.

A major advantage of zebrafish is that the larvae of zebrafish are optically transparent. At approximately 4.5 days post fertilization, the GI tract begins to contract regularly and spontaneously. Contractions of GI smooth muscle in zebrafish larvae can be easily observed under a dissecting microscope. Contraction frequency and coordination of motility serve as a quantitative and functional readout. Therefore, agents that modify GI contraction can be identified through direct visualization of the spontaneous contractions. Additional advantages of a zebrafish-based model system include the small size of zebrafish larvae, the ease of care and reproducibility, and the ability of zebrafish to respond to pharmacological manipulation similarly to humans.

Accordingly, in one embodiment, the zebrafish model system is used to study the gastrointestinal function in order to develop new pharmaceutical therapy for treating human gastrointestinal disorders related to motility. Specifically, the present invention provides a method for determining the effect of a candidate compound designed to treat a human gastrointestinal disorder by administering the compound to a zebrafish or zebrafish larvae and assessing the effect of the compound on motility of the GI tract of the zebrafish or larvae.

Motility of the zebrafish GI tract can be measured by counting the frequency of contractions of the GI tract by direct observation. Coordinated contractions that are propulsive in nature are measured in a functional manner by determining the rate of movement of the contents of the GI tract. For example, one can measure the transit time of certain materials, such as blue food dye, fluorescent markers such as polystyrene micro spheres labeled with FITC, or fluorescently labeled high molecular weight dextran.

Generally, the measurement involves loading zebrafish larvae with a marker dissolved in the fish media, quantifying the amount of the marker ingested by the larvae at time zero, and taking a second measurement of the amount of the marker in the larvae after a specific time interval. Digital imaging techniques can be used to quantify the relative brightness of fluorescence immediately after loading and twenty-four (24) hours later. A decrease in relative intensity after 24 hours will indicate that some of the fluorescent marker has been excreted as a result of propulsive GI contractions. Similar experiments can be performed using blue food dye, yielding similar results. However, the use of a fluorescent marker enhances the brightness of the dye within the GI tract, rendering the assay more sensitive.

Alternatively, a spectrophotometric assay can be used to measure the transit time of a marker. In this assay, larvae are loaded with a fluorescent marker. One half of the larvae are then sacrificed, and grounded using a mortar and pestle to release the marker into the media for measurement. The other half of the larvae are kept in a marker-free media for a specific time interval, and then the amount of the marker remaining in the larvae is measured and is compared to the measurement from the first half of the larvae. The difference between the two measurements represents the portion of the marker that is moved by the GI tract and eliminated.

It has been demonstrated by the present inventor that GI transit time decreases with age; for example, 11 day-old larvae pass fluorescent marker more quickly when compared with 7 day-old larvae. Quantification of GI transit time allows identification of compounds that either promote and enhance motility of the GI tract, or alternatively, decrease and inhibit motility of the GI tract.

Therefore, in another embodiment, the present invention provides a method for identifying a compound that either promotes or inhibits GI motility by administering the compound to a zebrafish or zebrafish larvae, and comparing the GI transit time of a material in the zebrafish or larvae with that of a control zebrafish or larvae (not treated with the test compound).

Identification of a desired effect of a specific compound, e.g., enhancing motility or inhibiting motility, permits the subsequent identification of the molecular mechanisms of GI functions and of the target of the compound.

In another embodiment, the present application is directed to the use of the zebrafish model system to assess the side effect profile of compounds that have been developed to treat a non-gastrointestinal disorder but may interfere with or alter gastrointestinal functions. In this embodiment, the present invention provides a method for assessing the side effects of a compound on gastrointestinal functions by administering the compound to a zebrafish and evaluating motility of the GI tract of the zebrafish.

In still another embodiment, mutant zebrafish are utilized to examine the role of a specific molecule or cellular component in the integrated GI function. For example, mutant zebrafish lacking specific signaling molecules, or specific anatomical features such as enteric neurons, pacemaker cells (ICC), or immune cells, are utilized in the examination. Therefore, the present invention provides a method for determining the function of a specific molecule or cellular component in the GI tract by obtaining a mutant zebrafish that lacks the molecule or cellular component, and determining the function of the molecule or cellular component by evaluating the GI tract of the mutant zebrafish.

For example, the receptor tyrosine kinase KIT is known to be expressed by ICC in the GI tract of humans, mice, rats, rabbits, guinea pigs, and dogs, and identification of ICC is made possible using this cell surface protein (termed c-KIT) as a marker. The zebrafish contains two genes, kita and kitb. The present inventor has shown, for the first time, that the zebrafish KIT protein is expressed within the zebrafish GI tract in the expected anatomical location for ICC. These findings are made by employing antibodies that are commercially available and developed based upon the human or murine KIT protein. The immunoreactivity detected in zebrafish based on antibodies developed against the human or murine KIT protein is also referred to herein as “KIT-like” immunoreactivity, and is believed to represent proteins encoded by either or both of the kita and kitb zebrafish genes.

It has been demonstrated by the present inventor that the spab5 mutant larvae, which lacks a functional kita gene, shows a decreased contraction frequency as well as a decrease in food transit time, indicating the importance of the zebrafish kita gene in the control of GI motility.

Additionally, it has been shown by the present inventor that KIT-like immunoreactivity has been observed in both wild type larvae and spab5 mutant larvae. Since the spab5 mutant zebrafish lacks a functional kita gene, the KIT-like immunoreactivity is believed to result from the expression of the kitb gene. As more robust KIT-like immunostaining has been observed in the wild type zebrafish compared to the spab5 mutant zebrafish, it is believed that both kita and kitb are expressed in the wild type larvae.

Moreover, mutant zebrafish can be used to examine the effects of compounds on each component of the GI tract, and to identify the specific target that compounds interact with. For example, the spab5 mutant sparse lacks a functional kita gene, which may represent a model with a partial ICC deficit, and the colourless mutant lacks enteric neurons. If a compound is a prokinetic in wild type zebrafish, but lacks effectiveness in the colourless mutant, then the target of the compound must be related to enteric nerve function. If the compound lacks effectiveness in the sparse mutant, then the target is on ICC or is influenced by ICC function. Therefore, the present invention provides a method for identifying the molecular or cellular target of a compound by determining the effect of the compound on the GI motility of a wild-type zebrafish, and the lack of the effect of the compound on the GI motility in a mutant zebrafish, thereby identifying the molecular or cellular component that is dysfunctional in the mutant zebrafish as the target of the compound.

It is also possible to measure the effects of identified compounds on rings or strips of tissues dissected from adult zebrafish GI tract. These experiments can further identify target suitability, as well as the possibility of tachyphylaxis after repeated application of a compound. Such experiments may be performed on tissues from both wild type and mutant fish.

The present invention is further illustrated by the following examples.

### EXAMPLE 1

Zebrafish larvae were maintained according to standard methods, described in the Zebrafish Book (Westerfield, 1993, The Zebrafish Book, University of Oregon Press, Eugene, Oreg.), in accordance with IUCAC guidelines. Wild type larvae and adults were obtained from Scientific Hatcheries, Huntington Beach, Calif. Larvae were also obtained from Mayo Clinic, Rochester, Minn., and Rosewell Park, Buffalo, N.Y. Spab5 mutant zebrafish were obtained from Washington University, St. Louis, Mo. Immunohistochemistry was performed on larvae and on freshly dissected adult GI tissue. Tissues were fixed in ice-cold acetone for 15 minutes, washed in PBS, and incubated in ACK2 (1:100) for 48 hours at 4° C. Tissues were washed and incubated with secondary antibody (1:200 dilution) for 24-48 hours at 4° C. Anti-desmin, anti HuC/HuD, (Molecular Probes), and anti cKIT (abcam) required 4% paraformaldehyde fixation overnight. Analysis was performed using a laser scanning confocal microscope (Zeiss LSM 510), and OptiGrid confocal system (Thales Optem, Rochester, N.Y.), and conventional fluorescence microscopy (Olympus BX51). Fluorescence images were digitized with a Spot RT digital camera (Diagnostic Instruments) and Image Pro Plus software (Media Cybemetics). Digital motion analysis to quantify larval GI contraction frequency was performed using a Spot Integra digital camera, collecting 7 frames/second, or a Nikon Coolpix 5400 (33 frames/sec).

### EXAMPLE 2

This Example describes an experiment designed to detect the levels of kita mRNA in the zebrafish GI tract.

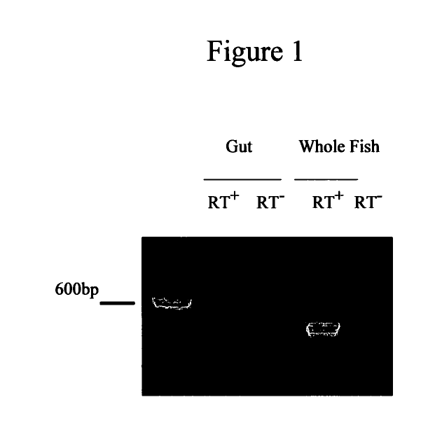

Primers were designed based on the zebrafish kita gene (AF153446) (62). RT-PCR was performed using total RNA isolated from the adult zebrafish GI tract, or whole zebrafish. A 640 bp product, the expected size of kita mRNA, was amplified using primers specific for the zebrafish kita gene (FIG. 1). These data indicate that kita mRNA is expressed in the GI tract of zebrafish, suggesting that KIT protein is expressed in the zebrafish GI tract.

### EXAMPLE 3

Pacemaker cells in human and other mammalian tissues are identified and distinguished from surrounding cells using antibodies to the KIT protein. This Example describes an experiment in which KIT-like immunoreactivity was detected in the zebrafish GI tract.

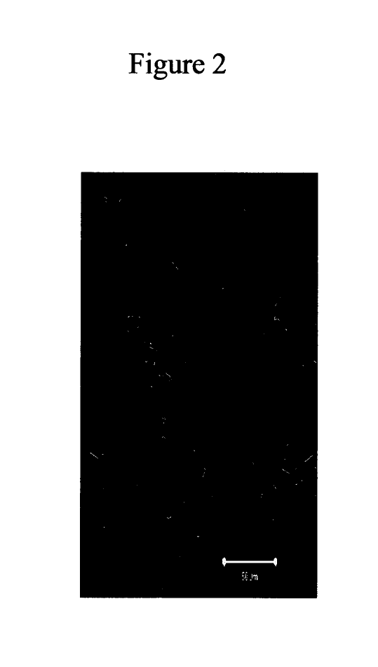

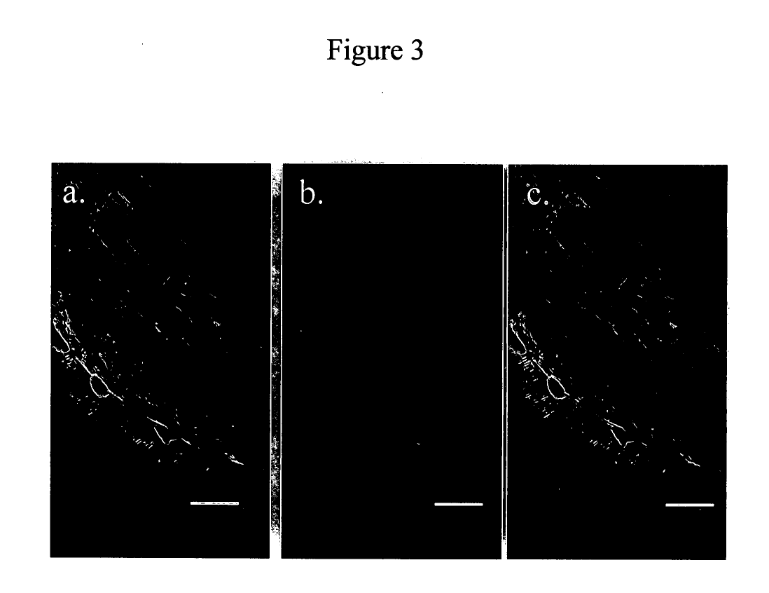

Immunohistochemistry was performed in an essentially identical manner as previously described for murine cell cultures (68). Briefly, after removal of GI tissue from adult zebrafish, tissues were fixed in ice-cold acetone, and incubated overnight at 4° C. with primary antibody, rinsed, and incubated with secondary antibody conjugated to CY3. These steps were identical to those used for identifying ICC within murine and guinea pig small intestine. FIG. 2 shows cells identified in the zebrafish GI tract using ACK2, a rat monocolonal antibody specific for an extracellular domain of the c-KIT protein (Chemicon). The image shows a stack-reconstructed confocal image resulting from compression of 27 optical sections, comprising approximately 13 μm. These data show for the first time the presence of putative ICC located in the tunica muscularis of the zebrafish GI tract.

### EXAMPLE 4

The zebrafish larvae have the strong advantage of transparency such that GI motility can be easily visualized. In this Example, zebrafish larvae GI tract was examined for KIT-like immunoreactivity.

Larvae (7-9 day old) were acetone-fixed and immunostained with ACK2. FIG. 2 shows a stack-reconstructed confocal image (panel a) resulting from compression of 41 optical sections, comprising approximately 20 μm. The image is a lateral view, oral is oriented to the left and dorsal is up. A transmitted light image shows the larvae (panel c). A network of cells encircling the GI tract was observed with KIT-like immunoreactivity. These data identified putative zebrafish ICC with KIT-like immunoreactivity in the zebrafish larvae. KIT-like immunoreactivity was also observed in the spab5 mutant sparse, a null-kit mutant, using the abcam anti-cKIT antibody and 4% paraformaldehyde fixation.

### EXAMPLE 5

ICC are similar in many ways to surrounding cells. For example, stellate ICC with branching process, enteric neurons, and macrophage cells share appearance; and bipolar ICC are morphologically similar to smooth muscle cells. ICC and smooth muscle cells, both of an origin in the mesoderm, share structural similarities, express similar ion permeable channels in the plasma membrane, and display similar rhythmic oscillations in membrane potential (41). This Example describes that ICC were identified in the zebrafish GI tract and were distinguished from neighboring cells.

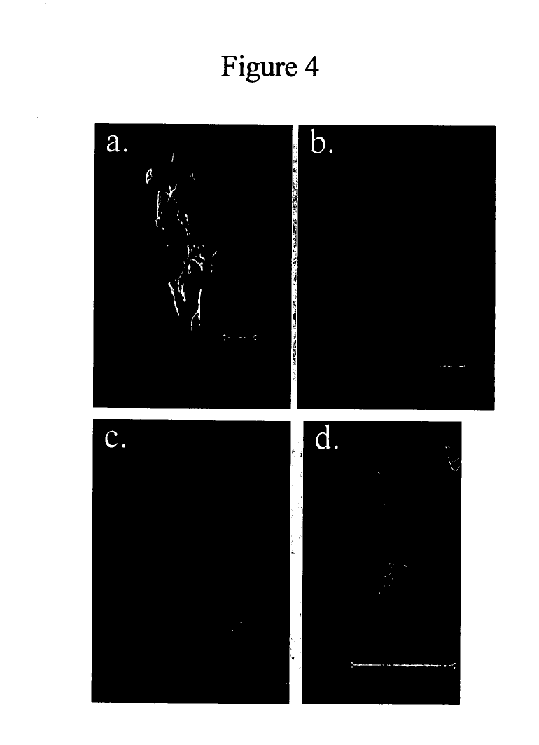

FIG. 4 shows fluorescence images from whole mounts of the adult zebrafish (panels a and b) and sections (panels c and d) of cells identified with the anti-KIT antibody ACK2 (panel a), the mitochondrial marker Mito Tracker red (panel b), the neuronal marker protein gene product 9.5 (panel c), and synaptophysin (panel d), which labels synaptic vesicle proteins. ACK2 is a rat monoclonal antibody raised against the mouse KIT protein, which has been used to identify and distinguish ICC from surrounding cells in mammalian GI tissue. MitoTracker Red accumulates in mitochondria in living cells and is retained after fixation. ICC are believed to contain many more mitochondria when compared to smooth muscle cells, and therefore mitochondria-specific fluorescent dye has been used to label ICC in the canine colon (92, 78, 47). Protein gene product (PGP) 9.5 is a rabbit polyclonal antibody raised against the human PGP 9.5 protein that is abundant in neuronal tissues. Synaptophysin is a rabbit polyclonal antibody that labels vesicular proteins in neurons.

The fluorescence images from intact tissues (FIG. 4) show that ACK2 labeled a specific cell population within the tunica muscularis of the zebrafish GI tract, and that MitoTracker red labeled a cell population with similar appearance, suggesting that these cells are ICC (panel a and b). Fluorescence images from sectioned tissues (panel c and d) show cells labeled with neuronal markers. Although MitoTracker red may not label ICC exclusively (it may also label neurons which have high mitochondrial density) and PGP 9.5 did not appear to label zebrafish neurons selectively in whole mounts, these data did show enteric neurons within the zebrafish GI tract. These data provide further support for the notion that KIT serves as a marker for the pacemaker cell in the zebrafish GI tract.

### EXAMPLE 6

This Example describes experiments performed to characterize rhythmic spontaneous contractions in zebrafish larvae.

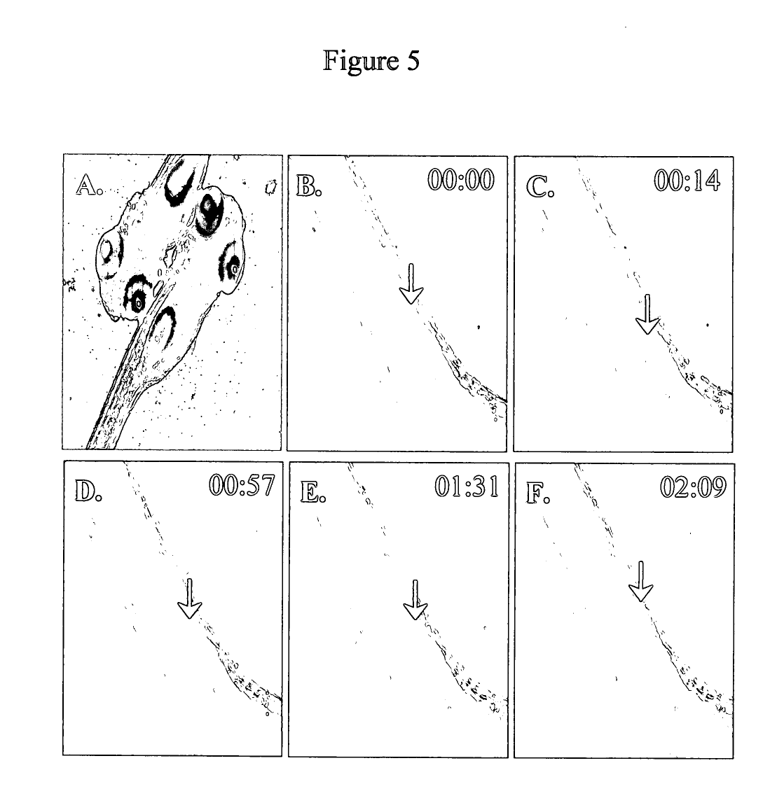

Larvae (10 dpf) were imaged after being fed with HFO food (Argent Redmond, Calif., particle size 30 μm) that was soaked in blue food-grade dye. The blue dye was clearly visible along the entire GI tract, from the wide proximal region near the swim bladder to the distal end opening (FIG. 5, panel A) after feeding for about 2 hours. FIG. 5, Panel B shows the initial frame, and each panel thereafter shows a single contraction where the lumen diameter was compressed, indicated by the arrowheads.

Images were collected using an inverted microscope and a Nikon Coolpix 5400 camera in video mode, collecting color images at 15 frames per second to a 1 Gbyte memory card. Contractions of the GI tract were clearly recorded using this method.

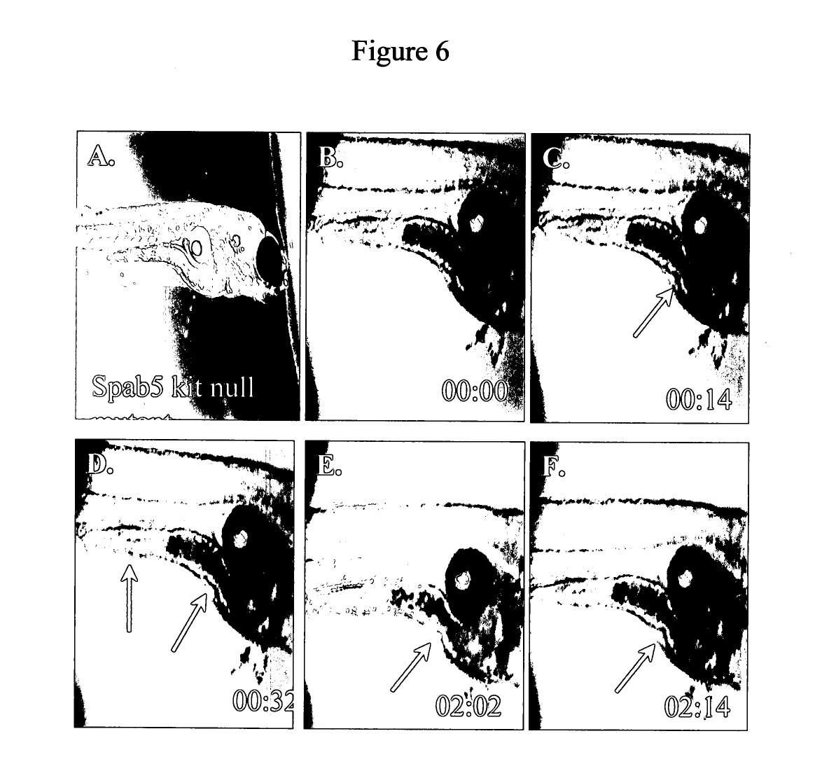

In contrast, the zebrafish kita null mutation sparse displayed irregular, disorganized, and reduced frequency spontaneous contractions (FIG. 6). The images shown in FIG. 6 were selected at peak contraction. The majority of spab5 mutants completely lacked spontaneous contractions. For this mutant contractions were observed at the position indicated by the arrow, and did not propagate.

These data demonstrate that contraction frequency and the organizational direction of the contractions in zebrafish larvae could be determined.

In sum, the above Examples show that KIT expression was detected in larvae and adult zebrafish at the mRNA and protein levels. Immunohistochemistry revealed KIT-like immunoreactivity in a cell population that lies between the circular and longitudinal muscle layers of the GI tract, and is highly similar in appearance to myenteric ICC in mammals. Null kita mutant larvae, spab5, displayed KIT-like immunoreactivity with different staining patterns, and were found to lack functional, coordinated GI contractions. These results suggest that putative ICC are necessary for spontaneous, coordinated contractions of GI smooth muscle. ICC identification in the zebrafish is an essential step toward development of a new zebrafish-based model system to elucidate the cellular and molecular details of gastrointestinal (GI) function, with an emphasis on motility. The zebrafish model system allows direct observation of GI motility in fully developed, intact larvae, and permits investigation of the mechanisms that control the coordinated contractions that give rise to GI function.

### EXAMPLE 7

This Example describes that imatinib mesylate inhibited ICC development in the zebrafish.

Background: The GI tract of the zebrafish has a cellular anatomy that is essentially similar to humans. The present inventor had previously verified expression of kita and kitb mRNA in the zebrafish GI tissues, and identified cells expressing KIT-like immunoreactivity in the myenteric plexus region of the zebrafish GI tract with morphological features of interstitial cells of Cajal (ICC). ICC were first identified at 7 dpf. The role of KIT-positive cells in GI motility of the Zebra fish had not been determined previous to this work.

Aim: Characterize normal spontaneous contractile activity in larvae and determine the functional role for ICC in zebrafish motility.

Methods: Zebrafish larvae are transparent and GI motility can be directly observed in intact larvae. Larvae (ages 5-14 dpf) were anesthetized and digital images of the intact larvae collected each second and the propagating intestinal contractions annotated. Contraction length was measured directly form the images and contraction velocity was calculated. The developmental time-course for propagating contractions was determined from 5 days post fertilization (dpf) until 14 dpf. In a second series of experiments imatinib mesylate (50 μM) was added to the larvae medium at age 5 dpf to block KIT function. Digital motion analysis was performed at 7 and 14 dpf.

Results: Spontaneous contractions of intestinal smooth muscles were first observed between 3 and 4 dpf. At 5 dpf spontaneous and disorganized contractions were observed in all larvae. Contractions were more organized by 7 dpf and with 65% propagated along the entire length of the intestinal tract. By day 14 dpf all contractions fully propagated. No change in contraction frequency occurred between 7 and 14 dpf, but propagation velocity increased as well as the mean propagation distance. Application of imatinib mesylate resulted in a decrease in the number of contractions. The remaining contractions were unorganized, shorter and slower, similar to contractions before appearance of ICC.

| | Larvae age | | |
|---|---|---|---|
| | | | 14 dpf (imatinib |
| | 7 dpf (n = 3) | 14 dpf (n = 3) | mesylat) (n = 3) |
| Total # | 17 | 17 | 7 |
| contractions/300 sec | | | |
| Fully Propagated | 11 | 17 | 2 |
| Velocity (μm/sec) | 14.3 ± 0.6 | 20.3 ± 2.3 | 10.2 ± 3.1 |
| Distance (μm) | 512.6 ± 54.8 | 663.0 ± 24.9 | 399.8 ± 67.2 |

These data show that propagating muscular contractions in the zebrafish intestine gradually develop between 5 and 14 dpf. Inhibition of Kit function blocks the development of propagating contractions. These data strongly suggest that ICC are necessary for propagating contractions in the zebrafish GI tract.

### EXAMPLE 8

The present inventor had identified ICC in the adult zebrafish GI tract. It is known that ICC development is required for the development of functional, coordinated GI motility that contribute to propulsion. This Example tested the hypothesis that ICC development is required for coordinated GI motility in the zebrafish. A pharmacological approach was utilized to block ICC development. Time lapse imaging of live larvae at various days post fertilization (dpf) was used to quantify GI motility. Specifically, the average velocity and the average length of a single contraction were measured, as well as the number of full propagated contractions per 300 second interval. The KIT receptor tyrosine kinase is required for ICC development. Gleevec® (imatinib mesylat), a specific KIT inhibitor, was used to block KIT function, and thereby prevent ICC development. GI motility in Gleevec treated larvae was used to determine the role of ICC on the development of coordinated contractions. Contractions in 7 dpf larvae were variable and few of fully propagated contractions were observed. Contractions in 14 dpf were consistent and all were fully propagated. Gleevec® treated 14 dpf larvae had few contractions which were variable and did not fully propagate. Quantification of GI motility variables is given in Table 1.

TABLE 1

| | 7 dpf | | 14 dpf | | 14 dpf (GLEEVEC) | |
|---|---|---|---|---|---|---|
| | Total | Average | Total | Average | Total | Average |
| Total # contractions | 17 | 5.67 ± 0.58 | 17 | 5.67 ± 0.58 | 7 | 2.3 ± 0.58 |
| Fully Propagated | 11 | 3.67 ± 0.58 | 17 | 5.67 ± 0.58 | 2 | 0.66 ± 0.58 |

| | | | |
|---|---|---|---|
| Velocity (μm/sec) | 14.3 ± 0.6 | 20.3 ± 2.3 | 10.2 ± 3.1 |
| Distance (μm) | 512.6 ± 54.8 | 663.0 ± 24.9 | 399.8 ± 67.2 |

These results show that coordinated GI motility develops between 7 and 14 dpf in zebrafish larvae, and Gleevec® inhibits this process. These data are consistent with the hypothesis that ICC are required for coordinated propagating GI contractions in the zebrafish.

### EXAMPLE 9

This Example describes the development of a new gastrointestinal motility assay using zebrafish.

Gastrointestinal (GI) motility disorders such as constipation and dyspepsia are common, but current treatment options are largely ineffective, primarily due to a limited understanding of GI motility. The broad objective is to develop a new zebrafish-based model system for GI motility. Muscular contractions of the GI tract may be directly observed in a zebrafish larva, and GI motility has been quantified as contraction frequency. However, counting contraction frequency does not discriminate between propulsive or mixing types of motility. The specific objective of this project is to develop an assay to specifically measure propulsion of luminal contents. Preliminary experiments showed that zebrafish larvae are able to ingest and excrete fluorescent microspheres (˜1 μm diameter) and we hypothesized that subsequent to an initial feeding of labeled microspheres the total fluorescence intensity should decrease with time. A decrease in fluorescence emission would indicate that ingested microspheres have been eliminated via propulsive motility. Fluorescence spectroscopy was used to quantify the fluorescence intensity as a function of time.

Total fluorescence emission averaged 62,630±23,780 counts per sec (n=5 samples, 5 larvae grouped per sample) immediately after ingestion in 11 day old larvae. One day later total fluorescence intensity averaged 53,277±6,333 counts per sec, a 15% decrease (n=5, P<0.05). These data suggest that propulsive motility resulted in microsphere expulsion, consistent with our hypothesis. Two prokinetic agents, erythromycin and 5-hydroxytryptamine (10⁻⁵ mg/ml), were used to validate the assay. One day after loading, the total fluorescence intensity decreased after application of erythromycin (69,782±10,423, n=5, P<0.09) and with 5 HT (86,820±21,850, n=5, P<0.003).

11 dpf zebrafish larvae ingest fluorescently labeled microspheres, and spectrophotometric analysis showed reduced fluorescence intensity one day after loading. These data support the hypothesis that total fluorescence emission in the larvae reduced with time. Validation of the assay was performed using two prokinetic agents, erythromycin and 5-hydroxytryptamine, that reduced fluorescence intensity by 23% and 53% respectively.

### EXAMPLE 10

This Example describes the identification of cells with Kit-like immunoreactivity in the Zebrafish gastrointestinal tract.

Background: At 4 days post fertilization the GI tract is fully developed with an intact enteric nervous system and rhythmic, spontaneous GI contractions are observed. In higher animals GI motility is initiated by the interstitial cell of Cajal (ICC), which expresses the kit receptor tyrosine kinase. Regulation of GI motility in the zebrafish has not been well studied, and the origin of spontaneous contractions in the gut of this animal is unknown. Zebrafish express an orthologue of the kit receptor but gene expression has not been studied in GI tissues.

Aims: The aim of this study was to determine if zebrafish express the kit gene within the tunica muscularis of the zebrafish GI tract.

Results: Total RNA was isolated from the GI tract of adult zebrafish, and from larvae. Specific primers were designed to the zebrafish kit gene (AF153446) and (AF153446). RT-PCR produced a 640 bp product, as predicted from the kit gene. Kit protein expression within the tunica muscularis of the GI tract was assessed by immunohistochemistry using the anti-murine c-Kit monoclonal antibody, ACK2. Intact larvae and whole GI tissues from adult zebrafish were acetone fixed and incubated with ACK2 for 24 hours at 4° C. Immunostained tissues were fixed in acetone, immunostained with ACK2 antibody, and examined with a laser scanning confocal microscope (LSM 510, Zeiss). A population of cells exhibiting KIT-like immunoreactivity was observed within the tunica muscularis of adult GI tissue. The Kit immunoreactive cells were similar in appearance to myenteric ICC identified in other species. KIT-like immunoreactivity was also observed in the GI tract of 9 day old zebrafish larvae. Stack-reconstruction of individual confocal images revealed a complete network of cells exhibiting KIT-like immunoreactivity encircling the GI tract.

Conclusion: These data show expression of the kit gene within the zebrafish GI tract and identification of kit-immunoreactive cells with ICC-like morphology in the zebrafish GI tract. ICC identification in the zebrafish is an essential first step toward development of a new zebrafish-based model system to elucidate the cellular and molecular details of gastrointestinal (GI) function, with an emphasis on motility.

### EXAMPLE 11

This Example describes that Kit-like immunoreactivity localizes in the myenteric plexus region of the zebrafish gastrointestinal tract.

Background: Gastrointestinal (GI) motility results from the coordinated actions of enteric neurons, interstitial cells of Cajal (ICC), and smooth muscle cells. The GI tract of the zebrafish has a cellular anatomy that is essentially similar to humans. Previous work by our lab has shown the presence of KIT-like immunoreactivity in the zebrafish GI tract. KIT-like immunoreactivity has been used extensively to identify ICC in the GI tract of other organisms. Characterization of the anatomical location of KIT-positive cells within the tunica muscularis of the GI tract and their relation to enteric nerves and smooth muscle is necessary to classify these cells as ICC subserving similar functions as ICC in other species.

Aims: To determine the localization of cells displaying Kit-like immunoreactivity and the relationship to enteric nerves and smooth muscle within the tunica muscularis of the zebrafish GI tract.

Methods: KIT protein expression within the zebrafish tunica muscularis was assessed by immunohistochemistry using the anti c-Kit polyclonal antibody, ab16832 (abcam). GI tissues dissected from larvae (7 days post fertilization) and from adult zebrafish were fixed in 4% paraformaldehyde overnight. Transverse sections (4-8 μM) were cut from paraffin-embedded tissues. Enteric neurons and smooth muscle cells were specifically identified using anti a tubulin and anti Hu C/D (Molecular Probes), and SM22 (abcam), respectively.

Results: Immunhistochemistry performed on tissue sections with anti a tubulin revealed a population of enteric neurons between the outer longitudinal layer and the inner circular layer of smooth muscle cells consistent with the enteric myenteric plexus. KIT immunolabeling showed a cell population with KIT-like immunoreactivity in the same region. Double labeling experiments revealed KIT positive cells adjacent to enteric neurons and smooth muscle within this layer. Intramuscular KIT immunoreactive cells were not observed, and no KIT immunoreactive cells were observed between the circular layer of smooth muscle cells and the mucosa. Kit-like immunoreactivity did not co-localize with smooth muscle or neural cell markers.

Conclusion: These data establish the presence of a cell population with Kit-like immunoreactivity in the myenteric plexus region within the zebrafish GI tract. Cells with Kit-like immunoreactivity were adjacent to enteric neurons, intercalated between enteric neurons and smooth muscle cells within the zebrafish GI tract. The anatomical arrangement of these Kit-positive cells strongly suggest that they are ICC.

### EXAMPLE 12

This Example describes that paralogs of the human Kit and steel factor genes are expressed within the gastrointestinal tract of zebrafish.

Gastrointestinal (GI) motility is essential for the proper digestion and absorption of nutrients in higher vertebrates, including humans. Observed GI systems require smooth muscle, enteric neurons, and interstitial cells of Cajal (ICC) in conserved anatomical organization to coordinate motility. ICC regulate contraction strength and frequency in human, mouse, and guinea pig GI smooth muscles. KIT, a receptor tyrosine kinase, is the traditional cellular marker for ICC in human, mouse, and guinea pigs. The KIT receptor and the Kit receptor ligand (steel factor) have been found to be necessary for ICC survival. Two paralogs of the human Kit gene, kita and kitb, and two paralogs of the human Kit ligand gene, kitla and kitlb, are present in zebrafish.

The aim of this investigation was to determine if the kit receptor is expressed within zebrafish GI tissues. Total RNA was isolated from GI tissues taken from wild type (AB) adult zebrafish, and expression was determined by reverse transcriptase PCR. For the first time we show that the zebrafish Kit receptors, kita and kitb, and Kit ligands, kitla and kitlb, are expressed in GI tissue. In separate experiments anti-KIT antibody specifically identified a cellular network within the myenteric plexus region of paraformaldahyde-fixed adult GI tissues. These data independently confirm the expression data. Expression of kita and kitb, and the presence of Kit-like immunoreactivity within the muscular layers of the zebrafish GI tract supports the hypothesis that ICC are present within the zebrafish GI tract.

### EXAMPLE 13

This Example describes GI motility in wild type and SPARSE mutant larvae. Gastrointestinal (GI) motility is the coordinated contractions of smooth muscles resulting in mixing and propulsion of material through the GI tract. GI motility is influenced by smooth muscle, enteric neurons, and interstitial cells of Cajal (ICC). New model systems for GI motility are needed because regulation of motility is poorly understood. The Kit receptor is required for ICC development and maintenance in mammalian model systems, and Kit mutations are associated with GI motility disorders. The objective of these experiments was to determine if the zebrafish kita-null mutant (spa b5) exhibits aberrant motility patterns when compared to wild type zebrafish. Contraction frequency was measured from 7 minute digital video recordings of 7 dpf larvae. Contraction frequency averaged 0.6±0.2 contractions/min (n=15) in wild type larvae (AB), and 0.33±0.16 (n=23) contractions/min in spa^{b5}. The apparent contraction intensity was also scored, with a score of 0 as no contraction and 2 as complete occlusion of the lumen. Contraction intensity averaged 1.2±0.41 for wild type, and 1.48±0.67 for spa^{b5} larvae. Two spa^{b5} larvae did not show GI contractions, and all wild type larvae showed GI contractions. A functional motility assay was developed to quantify GI motility contributing to propulsive movement. Larvae were fed FITC labeled microspheres, washed, and fluorescence intensity of the GI tract was digitally imaged. Larvae were re-imaged after 24 hours. Fluorescence intensity in a region of interest (ROI) located in the intestinal bulb decreased by 480±901 in wild type (n=27) and 1899±788 in spa^{b5} (n=12) larvae. Fluorescence intensity in an ROI located in the posterior GI tract increased by 203±300 in wild type (n=27) and decreased by 310±286 in spa^{b5} larvae (n=12). The data indicate that microspheres move out of the intestinal bulb and into the posterior GI tract. The kita null mutant spa^{b5} showed a decrease in fluorescence intensity in the intestinal bulb over time, indicating aboral movement. The decrease in fluorescence intensity of the posterior GI tract in spa^{b5} larvae was interpreted as evidence for elimination of microspheres, out of the posterior GI tract. In summary, spab5 mutant larvae exhibit fewer GI contractions, which are more effective for propulsive movement.

### EXAMPLE 14

This Example describes using a spectrophotometer assay that measures total fluorescence emission of fluorescent micro spheres that have been ingested by a zebrafish larvae.

After larvae spontaneously ingest the fluorescent micro spheres, which were suspended in the medium that the larvae are in, the larvae were washed and separated into 2 groups. The first group was sacrificed and mashed. The total fluorescence emission was measured from the ‘mashed’ larvae. The second group was sacrificed some time later. If GI motility is functional then some fraction of the micro spheres will have been excreted as a result of GI propulsive motility. The second group was sacrificed and mashed and total fluorescence emission was measured.

The difference between Group 1 and Group 2 represents the fraction of beads that has been excreted and this difference is a measure of functional GI propulsive motility.

### EXAMPLE 15

This Example uses digital imaging and image processing techniques to quantify coordinated GI motility. GI Motility refers to the movement of the GI tract resulting form muscular contractions. At the most basic level GI motility may be described by two functional terms: mixing and propulsive. Both types of motility are required for survival, and GI dysmotility is reflected by changes in the amount of either type of motility. These types of motility can be distinguished in time-lapse digital images. The present inventor previously measured the distance of each propagating contraction and calculated the velocity for each propagating contraction (distance/time).

This Example uses a technique, spatiotemporal mapping, that converts a series of images into a single image with dimensions of time and distance. This single image supplies data on movement, and therefore propagation contractions can be visualized and quantified. This technique has been used in the zebrafish (see Holmberg et al., J Exp Biol.; 210(Pt 6): 1084-91, Mar. 15, 2007). The present inventor has demonstrated that motility differs in wild type and mutant zebrafish larvae (Sparse mutant larvae that lack functional kita genes), and between wild type larvae and larvae treated with the tyrosine kinase inhibitor Gleevec. The data from this study are consistent with the hypothesis that the Interstitial cell of Cajal, required for coordinated motility patterns in humans, is present and serves a similar function in the zebrafish GI tract.

### EXAMPLE 16

### Experimental Procedures

Aquaculture

Wild-type and Sparse mutant zebrafish (ZFIN ID 960809-7 and 980202-7) were obtained from the Zebrafish international resource center and maintained according to standard guidelines in accordance with IACUC guidelines (Westerfield, Microsc Res Tech 47: 303-308, 1993). Wildtype long-finned golds (Scientific Hatcheries, Huntington Beach, Calif.) were also used for some preliminary experiments and no differences in immunohistochemical staining were observed when comparing strains. Fish were maintained at 28° C. in system water comprised of deionized water containing 240 mg/L Instant Ocean salts and 75 mg/L NaHCO₃ with 20% system water change each day (pH was adjusted to approximately 7.2, conductivity approximately 450 PPM). Zebrafish were fed 3 times daily, alternating Cyclopeeze (Argent, Redmond, Wash.) with live brine shrimp, and maintained on a 14-hr/10-hr light/dark cycle. Crosses were performed in the morning, and embryos were maintained in embryo medium in 400-ml beakers kept in a water bath set to 28° C. Larvae were fed hatch fry encapsulation, grade 0, beginning at 7 dpf, and live brine shrimp after 11 dpf (Argent, Redmond, Wash.).

Immunohistochemistry

Adult and larvae zebrafish were anesthetized in system water containing MS222 (3-aminobenzioc acid ethyl ester, Sigma Chemical Co., St Louis, Mo.) and sacrificed for immunohistochemistry. Intact larvae and freshly dissected adult GI tissues were fixed in freshly prepared 4% paraformaldehyde in phosphate buffered saline (Fisher) with pH adjusted between 7.3 and 7.4 for a minimum of 2 hr, but not longer than overnight. Immunostaining using the ACK2 antibody requires acetone fixation, and for these experiments larvae and tissues were fixed for 15 min in ice-cold acetone. Fixed tissues were washed 4 times in PBS containing 0.02% sodium azide. Larvae were carefully dissected such that the GI tract was separated from the body so that the head remained attached to the GI tract. Although time-consuming, removing the GI tract in this manner allowed direct access for antibody-antigen binding, producing consistent immunostaining. Nonspecific binding of primary antibody to tissues was minimized by incubation for at least 1 hr at 4° C. in blocking solution comprised of 10% normal donkey serum (NDS, Chemicon) and phosphate buffered saline containing 0.02% sodium azide, 0.1% Triton-X-100, and 0.05%-Tween 20 (PBS-TT). Primary antibodies were diluted in PBS-TT containing 5% normal donkey serum and were applied for 24-48 hr at 4° C. on an orbital platform. After washing 4 times in PBS-TT, tissues were incubated with appropriate secondary antibody conjugated to a fluorescent marker and diluted in PBS-TT containing 2.5% normal donkey serum for 24 hr at 4° C. on an orbital platform. Nonspecific immunoreactivity was assessed by immunostaining tissues or larvae in an identical manner but with the primary antibody omitted. The optimal concentration for each primary and secondary antibody was determined using serial dilutions. Antibodies were applied simultaneously during dual labeling experiments, followed by a PBS-TT wash and the simultaneous application of two appropriate secondary antibodies. Tissues were washed with PBS-TT and mounted on glass slides using Slow Fade medium (Invitrogen).

Paraffin-Embedded Sectioned Tissues

Adult fish were anesthetized in MS 222, decapitated posterior to the gills, tails were removed to aid fixative penetration and were immersed in freshly prepared 4% paraformaldehyde overnight at 4° C. on an orbital platform. The GI tract was subsequently dissected and washed with PBS followed by 70% ethanol. Tissues were dehydrated and paraffin embedded using a Tissue-Tek VIP E150 processor with 1-hr infiltration steps at 70% (1 step), 95% (2 steps), 100% (2 steps) ethanol, followed by xylene (3 steps) and paraffin infiltration (30 min, 4 steps) comprising a 12-hr schedule. GI tissues were sectioned into anterior, mid, and posterior portions and embedded in a paraffin block oriented for transverse sections. One piece of paraformaldehyde-fixed mouse GI tract (small intestine) was included in each block to serve as a positive control for immunostaining. Paraffin blocks were stored at 4° C. until sectioning. Specimen blocks were trimmed, soaked in ice water for 30 min, and sectioned at either 4 or 8 μm using a Reichart-Jung microtome. Sections were placed on slides (Superfrost plus, Fischer), baked at 56° C. for 1 hr, and stored at −20° C. until de-paraffinization.

Slides with sectioned tissues were placed on a 56° C. warming tray for 5 min, and then a xylene bath for deparaffinization (2 steps, 5 min). Tissue sections for hematoxylin and eosin staining were rehydrated using a graded alcohol series; 100% (2 steps, 3 min), 95% (2 steps, 3 min), followed by dIH₂O (1 step, 10 min). Tissue sections used for immunohistochemistry were similarly rehydrated followed by PBS-TT (1 step, 10 min). Preliminary immunostaining experiments using anti-Kit antibody resulted in a weak signal, and therefore antigen retrieval techniques were utilized to enhance specific immunoreactivity. Slides were immersed in antigen retrieval solution (Abcam) at 100° C. for 20 min., allowed to cool to room temperature in the same solution, and rinsed in PBSTT. Immunostaining was performed at room temperature. Tissue sections were incubated in blocking solution (1 hr), primary antibody solution (1 hr), and secondary antibody solution (1 hr). Optimal dilutions were determined for each primary and secondary antibody. Wash steps using PBS-TT were performed after the primary and secondary antibody incubations. Sections were cover slipped after application of Slowfade (Invitrogen). Fluorescence and transmitted light imaging. Tissues were examined with conventional light and fluorescence microscopy using an Olympus BX51 microscope equipped with an Optiscan z-axis controller (Prior Scientific) and supported on an anti-vibration table (Technical Manufacturing Corporation, Peabody, Mass.). Images were captured using a Spot RT digital camera (Diagnostic Instruments) or a Retiga EXi digital camera (QImaging) using Image Pro Plus software version 5.0 (Media Cybernetics). High-resolution images were collected using an Opti grid structured light imaging system (QIOPTIC, Rochester, N.Y.). A 100-W mercury lamp was used for epi-fluorescence illumination with appropriate excitation-emission filter sets for each fluorophore. A laser-scanning confocal microscope (model LSM 510, Zeiss) was used for images. Images were reconstructed from confocal stacks of z-series scans as indicated.

Functional GI Motility Measurements

GI motility was assayed using live larvae that were incubated in embryo medium containing blue food dye to enhance contrast of the GI tract lumen. The dye did not effect larvae survival, heart rate, or GI tract contraction frequency. Larvae were fully anesthetized and mounted laterally in 1.2% agar to permit an optimal viewing of the GI tract, and to prevent drift during filming. A drop of anesthetic was placed on the agar to keep it from drying out and to keep larvae anesthetized during filming. Spontaneous GI contractions were recorded continuously for 10 min using a Cannon Optura Xi digital video camera and converted to digital format (Pinnacle Studio AV/DV). Contractions were counted manually at a single position in the mid-intestine during replay of digitized video at an increased rate.

Reverse Transcriptase PCR

Experiments were done to determine if the zebrafish Kit receptors kita and kitb, and the Kit ligand (Steel factor) kitla and kitlb, are expressed in GI tissues. Total RNA was prepared from freshly dissected GI tissues of adult wild-type zebrafish, and from whole zebrafish (used as a positive control), using the RNeasy kit (Qiagen, Chatsworth, Calif.). First-strand synthesis was performed using random decamer primers. Gene-specific PCR was performed using Taq Pfx (Invitrogen) with 1 μl of the reaction mixture from the first-strand synthesis and primers specifically designed for each gene, and run for 35 cycles. Optimal magnesium concentration and primer annealing temperature were independently determined for each primer set. Amplification products were resolved on a 2.5% agarose gel.

Results

Kit expression in the zebrafish GI tract was identified using a rabbit polyclonal antibody specifically designed to recognize C terminal amino acids 961-976 of human c-Kit. A continuous and extensive network of cells displaying Kit-like immunoreactivity was observed within the muscular layers of paraformaldehyde-fixed adult zebrafish GI tract. Two distinct populations of cells were observed in separate layers of whole mount tissue. One layer displayed elongated cell bodies with multiple branching processes forming a loose but regular network pattern, and a second layer was comprised of bipolar or simple bifurcating cells located deeper within the circular muscle layer. Images were taken from mid-intestinal segments, as classified by (Wallace et al., Mech Dev 122: 157-173, 2005). Substantial differences in distribution of Kit-positive cells in each segment were not observed.

Spontaneous contractions of the zebrafish GI tract begin at 4 dpf, coinciding with the development of enteric neurons, but the possibility for ICC contributing to the development of rhythmic contractions has not been explored (Kelsh et al., Development 127: 515-525, 2000; Shepherd et al. Development 131: 241-249, 2004; and Wallace et al. Dev Cell 8: 717-726, 2005). Kit expression was identified in the GI tract of paraformaldehyde fixed zebrafish larvae. The GI tract was carefully dissected from larvae prior to immunostaining to maximize antibody penetration of intact tissues. Single confocal sections were taken midway through the digestive tube for 7, 11, and 20 dpf larvae. Cells displaying Kit-like immunoreactivity were observed in the outer layer of the tunica muscularis, indicated by arrowheads. Kit expression appeared to increase from 7 to 11 dpf, and it was possible to identify an apparent network of Kit-positive cells at 20 dpf. Kit-like immunoreactivity was not observed prior to 7 dpf. It was not possible to separate the mucosa from the tunica muscularis in adult or larvae GI tissues, which contributed to high background staining and prevented complete confocal stack reconstructions of the full-thickness digestive tract in larvae. The ACK2 rat monoclonal antibody that has been widely used to identify mouse ICC also specifically identified cellular networks in acetone-fixed adult and larvae zebrafish GI tissues.

Two types of Kit-positive cells are observed at high magnification: one branching cell with prominent nuclei and thinner bipolar cells. The anatomical position of these cells was determined in transverse sections of adult zebrafish GI tissues. Hematoxilyn and eosin stained transverse sections show that the outer longitudinal and inner circular smooth muscle layers are approximately 2 and 4-5 cell layers thick, respectively. Kit-like immunoreactivity was consistently observed in two distinct layers of cells in transverse sections of adult GI tissues. The outer layer of cells with Kit-like immunoreactivity appeared more dense and continuous, and a second thinner layer of Kit-positive cells was observed closer to the lumen. The inner layer was discontinuous and oriented in parallel with the circular smooth muscle cells. Both layers of Kit-positive cells were observed in anterior, mid, and posterior intestinal segments. The anatomical position of Kit-positive cells was also determined using transverse sections of zebrafish larvae. A single continuous layer of Kit-positive cells within the tunica muscularis of the GI tract was observed in 13 dpf larvae.

The relative anatomical positions of Kit-positive cells, enteric neurons, and smooth muscle cells in the zebrafish GI tract was examined further for comparison with established mammalian model systems. A pan-neuronal antibody, anti HuC/D, was used to identify cell bodies, and anti-acetylated α-tubulin antibody was used to identify neural processes. Composite images resulting from dual labeling experiments with whole mounted tissue show that cells displaying Kit-like immunoreactivity are in the same area as neuronal cell processes, labeled with acetylated α-tubulin antibody. Composite images of transverse sections of Kit-positive cells and neural cell processes located in the myenteric plexus region. Similarly, dual labeling with anti Hu C/D and anti-Kit antibody shows neural cell bodies located near Kitpositive cells in whole mounted tissues, and in the myenteric plexus region in transverse sections. Enteric nerves were only occasionally observed to extend deep into the circular muscle layer near the thin inner layer of Kit-positive cells. Dual labeling with anti-SM22 antibody to detect smooth muscle cells and anti-c-Kit antibody identified longitudinal and circular smooth muscle layers, and Kit-positive cells located between the layers in the whole mounted tissue. Smooth muscle cells were identified using an anti-Desmin antibody in transverse sections. Desmin is an intermediate cytoskeleton filament protein expressed by GI smooth muscles. Composite images of transverse sections dual labeled with anti-Desmin and anti-Kit antibody show one dense layer of Kit-positive cells positioned between the longitudinal and circular muscle layers and a second thin layer of Kit-positive cells near the innermost circular smooth muscle cells.

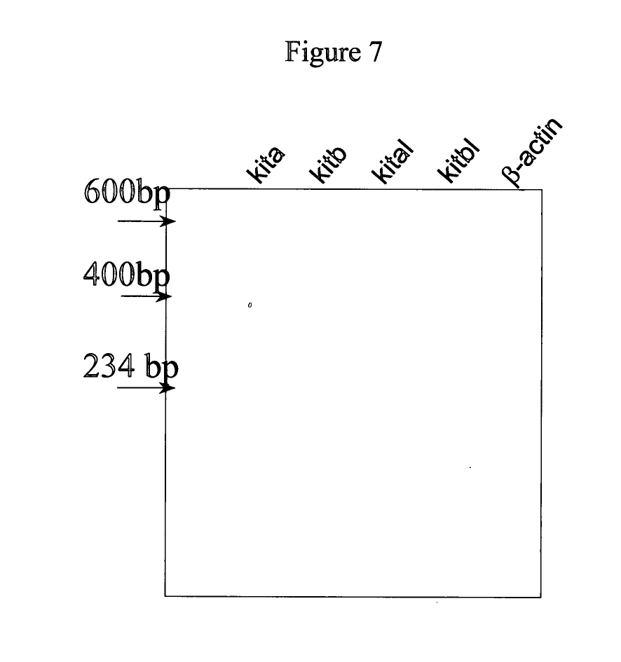

Expression of the zebrafish orthologs of mammalian c-Kit was verified by determining mRNA expression for the kita and kitb genes within GI tissues. Reverse transcriptase PCR was performed on cDNA prepared from total RNA isolated from adult zebrafish GI tissues. The presence of mRNA for two known orthologs of the c-Kit receptor, kita and kitb, as well as orthologues of c-Kit ligand, kitla and kitlb, were determined using specific primer sets for each gene, and for β-actin as a positive control (see Table 1). Primers were intron-spanning to rule out the possibility of genomic contamination. Products of the expected size for kita, kitb, kitla, and kitlb were amplified (FIG. 7). The identity of the bands was confirmed by sequencing. These data show for the first time the presence of mRNA encoding kita, kitb, and the ligands for these receptors, kitla and kitlb, in the zebrafish GI tract.

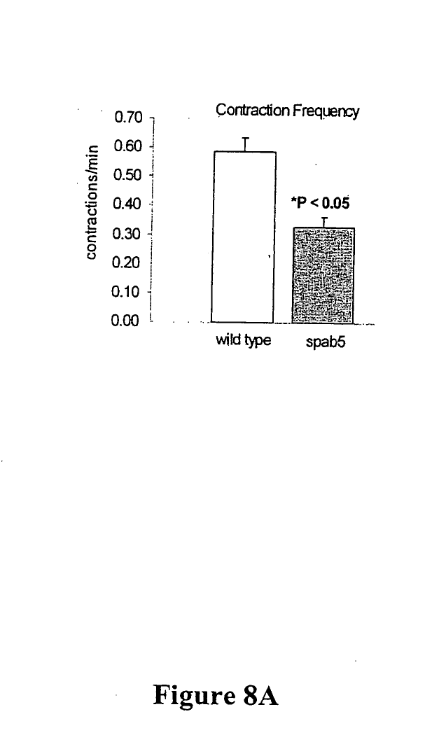

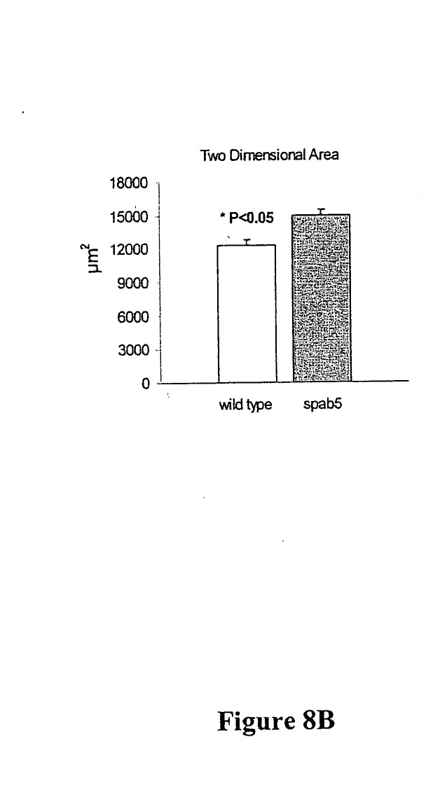

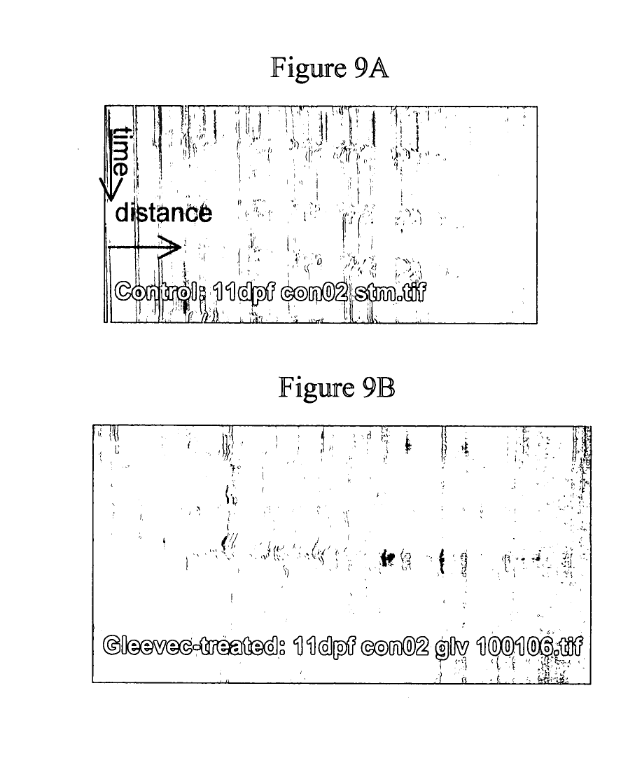

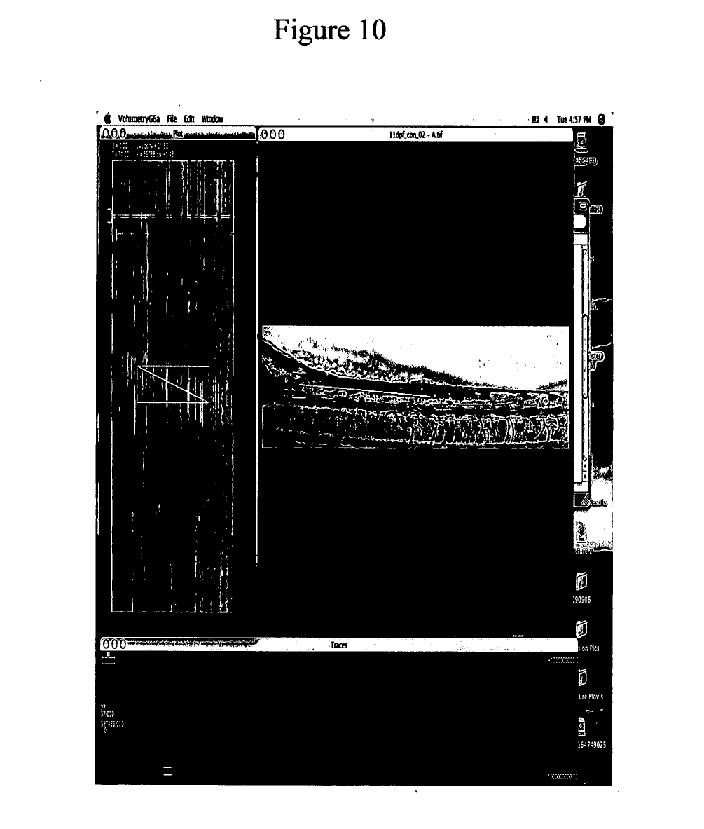

Development of ICC requires functional c-Kit signaling during embryogenesis in the mouse model, and also to maintain ICC in adults (Maeda et al., Development 116: 369-375, 1992; Torihashi et al., Cell Tissue Res 280: 97-111, 1995; and Beckett et al., Development (in press), 2006). The present inventor examined homozygous Sparse mutants spab5 (ZDB-FISH-980202-47) for functional differences in GI motility and for differences in appearance of the GI tract to determine a role for the kita gene. Contraction frequency was reduced in Sparse mutants compared to wild-type 7 dpf larvae. Contraction frequency averaged 0.59+/−0.05 (mean +/−standard error, n=20) contractions per minute in wild-type larvae, and 0.33+/−0.03 (mean +/−standard error, n=23, P<=0.05) contractions per minute in Sparse mutants (FIG. 8). Inactivation of c-Kit by injection of the neutralizing antibody ACK2 in the mouse resulted in distension of the stomach, small intestine, and colon, and, therefore, the size of the GI tract in Sparse mutants was compared to wild0type larvae (Maeda et al., Development 116: 369-375, 1992; and Torihashi et al., Gastroenterology 117: 140-148, 1999). Sparse and wildtype larvae were incubated for 1 hr in media containing FITC-labeled dex-tran, anesthetized, and washed. The lumen of the GI tract was observed using fluorescence microscopy. The cross-sectional area of the lumen was outlined from fluorescent images and measured using Image Pro Plus software (Media Cybernetics, version 5.0). The size of the GI tract in 7 dpf Sparse mutants was larger compared to 7 dpf wild-type larvae, 12,331+/−467 μM and 14,985+/−483 μm², respectively (mean +/−standard error, n=20 wild-type, 12 Sparse, P<=0.05). Adult Sparse zebrafish consistently exhibited a distended GI tract, which was most apparent in the intestinal bulb. Cells displaying Kit-like immunoreactivity were observed in Sparse mutant larvae and in adult GI tissues, with a similar density as wild-type zebrafish.

TABLE 1

| | | Expected |
|---|---|---|
| Target | Sequence | size (bp) |
| kita | F: GTT ATC CCA CTC CTC AGA TCA AGT | 564 |
| (AF153446) | (SEQ ID NO: 1) | |
| | R: TCA CAG CTA CAG TCATCA CAG TGT | |
| | (SEQ ID NO: 2) | |
| kitb | F: GGG AGG AAT CAC CAT CAG AA (SEQ | 234 |
| (DQ072166) | ID NO: 3) | |
| | R: CTC AGG TGG AAA TCGTGG TT (SEQ | |
| | ID NO: 4) | |
| kital | F: CACAGTTGCTGCCTATTCCA (SEQ ID | 580 |
| (AY929068) | NO: 5) | |
| | R: GGTGAGGAGCCACCTGAGAT (SEQ ID | |
| | NO: 6) | |
| Kitbl | F: GGC TGC ATT TGA ACC TGT ATC C | 542 |
| (AY929069) | (SEQ ID NO: 7) | |
| | R: GTG TCT GCA CAC CCTAAA GAA | |
| | TCC (SEQ ID NO: 8) | |
| β actin | F: GAT ACG GAT CCA GAC ATC AGG | 580 |
| (BC067566) | GTG TCA TGG TTG GTA (SEQ ID | |
| | NO: 9) | |
| | R: GAT ACA AGC TTA TAGCAG AGC TTC | |
| | TCC TTG ATG (SEQ ID NO: 10) | |

What is claimed is:

1. A method for determining the effect of a candidate compound designed to treat a gastrointestinal disorder in human, comprising administering the compound to a zebrafish or zebrafish larvae and assessing the effect of the compound on motility of the GI tract of the zebrafish or larvae.

2. A method for identifying a compound that either promote or inhibit GI motility, comprising administering the compound to a zebrafish or zebrafish larvae, and comparing the GI transit time of a material in the zebrafish or larvae with that of a control zebrafish or larvae.

3. The method of claim 2, wherein said material is a food dye or a fluorescently labeled marker.

4. A method for assessing the side effects of a compound on gastrointestinal functions, wherein the compound has been developed for treating a non-gastrointestinal disorder, comprising administering the compound to a zebrafish or zebrafish larvae, and evaluating the motility of the GI tract of the zebrafish or zebrafish larvae.

5. A method for determining the function of a specific molecule or cellular component in the GI tract, comprising obtaining a mutant zebrafish that lacks the molecule or cellular component, and evaluating the GI tract of the mutant zebrafish thereby determining the function of the molecule or cellular component.

6. A method for identifying the molecular or cellular target of a compound, comprising determining the effect of the compound on the GI motility of a wild type zebrafish, and determining the lack of the effect of the compound on the GI motility in a mutant zebrafish, thereby identifying the molecular or cellular component that is dysfunctional in the mutant zebrafish as the target of the compound.
